(19) United States
(12) Patent Application Publication
(10) Pub. No.: US 2022/0175355 A1
(43) Pub. Date: Jun. 9, 2022

(54) MEDICAL MATERIAL

(71) Applicant: GUNZE LIMITED, Ayabe-shi, Kyoko (JP)

(72) Inventor: Saki OKUMURA, Ayabe-shi (JP)

(73) Assignee: GUNZE LIMITED, Ayabe-shi, Kyoto (JP)

(21) Appl. No.: 17/436,319

(22) PCT Filed: Mar. 26, 2019
(86) PCT No.: PCT/JP2019/012749

§ 371 Date: Sep. 3, 2021

(30) Foreign Application Priority Data
Mar. 4, 2019 (JP) 2019-038117

(51) Int. Cl. A61B 17/00

(52) U.S. Cl. CPC A61B 17/0057; A61B 2090/3966; A61B 2017/00592; A61B 2017/00606

(57) ABSTRACT

A defect hole closing material achieves less invasive treatment for atrial septal defect with no or little possibility of problems in the late post-treatment period. A defect hole closing material includes two tubular bodies (a first tubular portion and a second tubular portion) made of a bioabsorbable material and having a mesh structure; and a substantially middle portion between the two tubular bodies. The first tubular portion is provided with a radiopaque material, the second tubular portion is provided with a radiopaque material, and the substantially middle portion is provided with a radiopaque material. In catheterization for atrial septal defect, when the defect hole closing material is pushed out of a catheter until the radiopaque material reaches an exit of the catheter in a fluoroscopic X-ray image obtained by X-ray imaging of a site including the defect hole closing material, only the second tubular portion increases in tube diameter while the first tubular portion does not increase in tube diameter.

### TECHNICAL FIELD

The present invention relates to a medical material for treating a defect hole in biological tissue, and particularly relates to a medical material configured to be set in a catheter, sent to a treatment site through a blood vessel, and placed in a living body.

### BACKGROUND ART

The heart of a human is divided into left and right chambers by tissue called the septum, and each of the left and right chambers has an atrium and a ventricle. That is, the heart is composed of two atria and two ventricles, i.e., right atrium, right ventricle, left atrium, and left ventricle. With regard to the heart having such a structure, atrial septal defect (ASD) is known, which is a defect wherein, due to a disorder of development in the fetal period, there is a congenital hole called a defect hole in the atrial septum separating the right atrium and the left atrium.

Treatment for atrial septal defect can be performed by the following two methods. One is a surgical operation performed by opening the chest, and the other is catheterization using an occluder without opening the chest.

A surgical operation (patching operation) involves using cardiopulmonary bypass, opening the chest, and closing the defect hole with a patch. Catheterization involves setting an occluder in a catheter, inserting the catheter into a blood vessel, sending the catheter to a target position (defect hole), and then releasing the occluder to place it in the body. The catheterization is to close a hole without opening the chest, by sending a small jig (device) called an occluder, folded in an elongated shape, from a vein (femoral vein) at the groin to the position of the hole in the atrial septum. The catheterization is advantageous in that the treatment can be performed merely by making a tiny skin incision (a few millimeters) in the groin (inguinal region), which is an inconspicuous area, without having to perform open chest surgery requiring general anesthesia.

Japanese Unexamined Patent Application Publication (Japanese translation of PCT International Application) No. 2008-512139 (Patent document 1) discloses an assembly (occluder) for use in catheterization for atrial septal defect. This assembly seals a passageway (defect hole) in the heart. The assembly includes: a closure device for sealing the passageway in the heart including a first anchor adapted to be placed proximate a first end of the passageway, a second anchor adapted to be placed proximate a second end of the passageway, and a flexible elongate member adapted to extend through the passageway and connect the first and second anchors, the second anchor capable of movement relative to the flexible elongate member to vary a length of the flexible elongate member between the first and second anchors; and a delivery system for delivering the closure device to the passageway in the heart, the delivery device being configured to move within a lumen of a guide catheter and including a wire configured to control movement of the second anchor along the flexible elongate material.

Patent document 1 also discloses that a patent foramen ovale (PFO) closure device (occluder) includes a left atrial anchor, a right atrial anchor, a tether, and a lock, and that the left atrial anchor, the right atrial anchor connected to the left atrial anchor via the tether, and the lock will remain in the heart to seal the PFO.

### RELATED ART DOCUMENTS

### Patent Documents

[Patent document 1] Japanese Unexamined Patent Publication (Japanese translation of PCT International Application) No. 2008-512139

### DISCLOSURE OF THE INVENTION

### Problems to be Solved by the Invention

A patching operation has an issue in that it involves usage of cardiopulmonary bypass, is highly invasive, and therefore requires long hospitalization. Catheterization is preferable because it does not involve usage of cardiopulmonary bypass, is less invasive, and therefore requires only short hospitalization.

As disclosed in Patent document 1, the left atrial anchor and the right atrial anchor remain in the heart. Each of the left and right atrial anchors includes one or more arms, which extend radially outward from a hub. The arms are preferably formed from a rolled sheet of binary nickel titanium alloy. A defect hole is to be closed by extending the left atrial anchor and the right atrial anchor in a living body; however, once the extension of the anchors has been started, it is difficult to bring the anchors into their original state. The anchors are to be folded by means of a dedicated takeout device which has a complicated structure and which is difficult to operate from outside the living body, as disclosed in Patent document 1.

However, for example, in the event that an anchor has accidentally been caught in biological tissue within an atrium and damaged the biological tissue, there may be cases where there is not enough time to fold the anchor using such a dedicated takeout device. In such a case, there is no other choice but to perform open chest surgery immediately. Under such circumstances, the patient will end up with highly invasive open chest surgery, which is an issue.

There is another issue in that a defect hole occluder made of metal will remain in the body for the whole life and that some problem may occur in the late post-treatment period.

The present invention was made in view of the above-mentioned issues of the conventional techniques, and its object is to provide a medical material which makes it possible to perform less invasive catheterization capable of releasing and placing the medical material at a treatment site inside a living body with easy operation without a complicated structure and which is unlikely to cause problems in the late post-treatment period even when remaining in the body.

### Means of Solving the Problems

In order to attain the above object, a medical material according to an aspect of the present invention provides the following technical means.

Specifically, a medical material according to the present invention is a medical material comprised of a tubular body that has a mesh structure formed of a linear material, wherein: the medical material has a shape in which a substantially middle portion of the tubular body is smaller in tube diameter than other portions of the tubular body; the medical material has a first tubular portion with a first end and a second tubular portion with an opposite end which are arranged with the substantially middle portion therebetween, the first end and the opposite end being opposite ends of the medical material in a longitudinal direction of the tubular body; and the first and second tubular portions and the substantially middle portion are provided with a radiopaque material.

It is preferable that the medical material can be configured such that the medical material includes an elastic member which has opposite ends respectively engaged with a linear material at the first end and a linear material at the second end and which passes through the first tubular portion and the second tubular portion from the first end to the second end via the substantially middle portion.

It is more preferable that the medical material can be configured such that, when the elastic member is in a contracted state, the first end and the second end are close to each other with the substantially middle portion therebetween and the foregoing other portions increase in tube diameter.

It is more preferable that the medical material can be configured such that, when the elastic member is in a contracted state, the foregoing other portions increase in tube diameter to a size corresponding to a defect hole to be closed with the medical material.

It is more preferable that the medical material can be configured such that, when the elastic member is in an extended state, the first end and the second end are away from each other with the substantially middle portion therebetween and the foregoing other portions decrease in tube diameter.

It is more preferable that the medial material can be configured such that, when the elastic member is in an extended state, the foregoing other portions decrease in tube diameter to a size corresponding to a catheter in which the medical material is to be contained.

It is more preferable that the medial material can be configured such that the elastic member is a coil spring having a smaller diameter than the tube diameter of the substantially middle portion.

It is more preferable that the medial material can be configured such that the shape is a sandglass shape, a figure-of-eight shape, or a double spindle shape.

It is more preferable that the medial material can be configured such that the linear material is a bioabsorbable material.

It is more preferable that the medial material can be configured such that a porous tubular layer composed of nonwoven fabric, a sponge, a film, or a composite thereof, each made of a bioabsorbable material, is disposed on an inner surface of the tubular body.

### Effects of the Invention

A medical material according to the present invention makes it possible to perform less invasive catheterization capable of releasing and placing the medical material at a treatment site in a living body with easy operation without a complicated structure. Furthermore, the medical material according to the present invention is unlikely to cause problems in the late post-treatment period even when remaining in the body.

### BRIEF DESCRIPTION OF THE DRAWINGS

FIG. 1 is an overall view of a defect hole closing material 100 as an example of a medical material according to the present invention (when a coil spring 140 is in a contracted state).

FIG. 2A is an overall view of the defect hole closing material 100 as an example of a medical material according to the present invention (when the coil spring 140 is in an intermediate state).

FIG. 2B is a perspective view of FIG. 2A.

FIG. 3 is an overall view of the defect hole closing material 100 as an example of a medical material according to the present invention (when the coil spring 140 is in an extended state).

FIG. 4 is an overall view of the defect hole closing material 100 as an example of a medical material according to the present invention (when the coil spring 140 is in the contracted state and in the extended state).

FIG. 5A is a partial side view of the defect hole closing material 100 in FIG. 2A.

FIG. 5B is a cross-sectional view taken along A-A in FIG. 5A.

FIG. 6 is a conceptual view in which the defect hole closing material 100 as an example of a medical material according to the present invention is used in catheterization for atrial septal defect.

FIG. 7 is an enlarged view (1) of a part B in FIG. 6 illustrating a procedure of catheterization.

FIG. 8 is an enlarged view (2) of the part B in FIG. 6 illustrating the procedure of catheterization.

FIG. 9 is an enlarged view (3) of the part B in FIG. 6 illustrating the procedure of catheterization.

### BEST MODE FOR CARRYING OUT THE INVENTION

The following description discusses a medical material according to the present invention in detail with reference to the drawings. Although the following description discusses a defect hole closing material for use in catheterization as an example of the medical material according to the present invention, the medical material is suitably applicable also to closure of other openings or passageways including, for example, other openings in the heart such as ventricular septal defect and patent ductus arteriosus and openings or passageways in other parts of a living body (for example, stomach) such as arteriovenous fistula. As such, the defect hole closing material according to an embodiment of the present invention is not limited to be used for the closure of a hole of atrial septal defect.

Moreover, although the description in the following embodiment is based on the assumption that a mesh structure of a defect hole closing material (occluder) 100 is knitted or woven from bioabsorbable fiber (an example of a linear material), the present invention is not limited thereto. It is only necessary that the defect hole closing material enable catheterization to close a defect hole in a living body, and its mesh structure may be knitted or woven from a linear material other than the bioabsorbable fiber, provided that the material has first to third features described later and achieves first to third effects described later. Such a linear material preferably has a certain degree of hardness to achieve form retaining property (shape retaining property).

[Configuration]

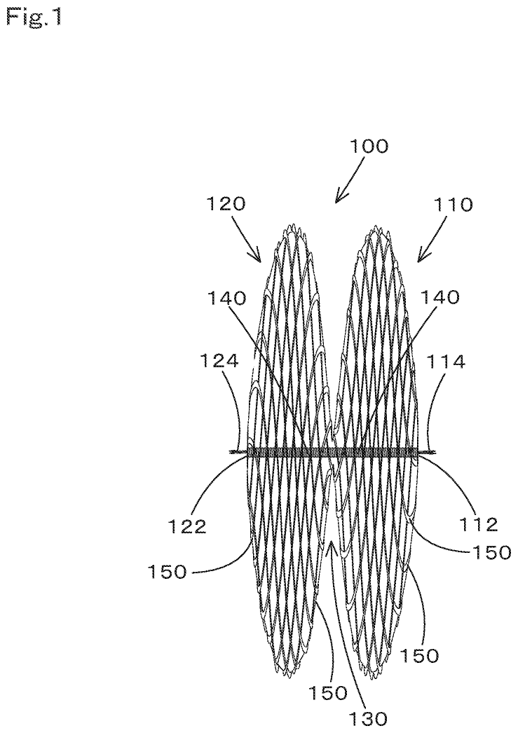

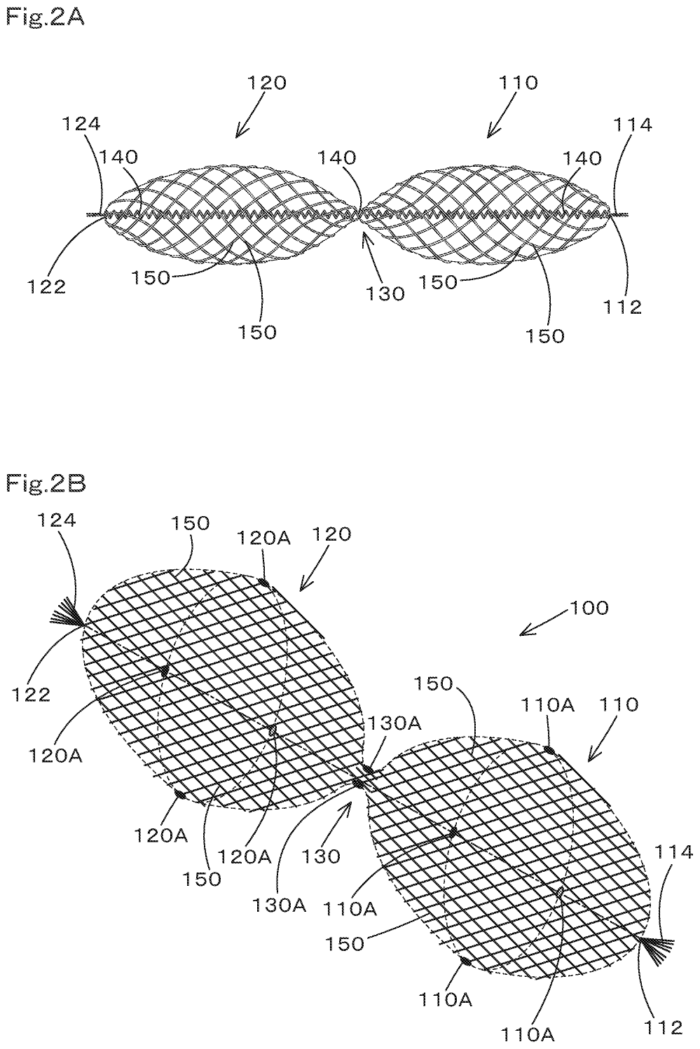

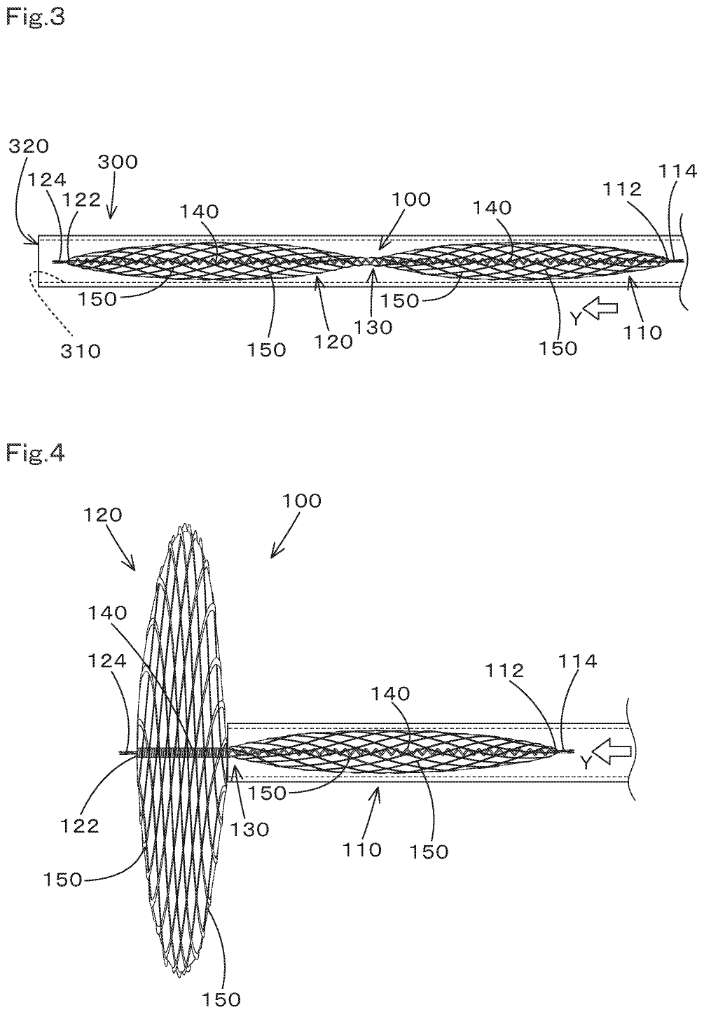

FIG. 1 is an overall view of the defect hole closing material 100 according to the present embodiment (when a coil spring 140 is in a contracted state), FIG. 2A and FIG. 2B are each an overall view of the defect hole closing material 100 (when the coil spring 140 is in an intermediate state), FIG. 3 is an overall view of the defect hole closing material 100 (when the coil spring 140 is in an extended state), and FIG. 4 is an overall view of the defect hole closing material 100 (when the coil spring 140 is in the contracted state and in the extended state). FIG. 3 illustrates the defect hole closing material 100 which is entirely contained in a catheter 300, and FIG. 4 illustrates the defect hole closing material 100 which is half (a first tubular portion 110) contained in the catheter 300. When the defect hole closing material 100 entirely contained in the catheter 300 (in the space defined by an inner wall 310) illustrated in FIG. 3 is pushed from the first tubular portion 110 side in the direction indicated by an arrow Y so that a second tubular portion 120 is pushed out through an opening 320 of the catheter 300, the state of FIG. 4 results. When the first tubular portion 110 is further pushed in the direction indicated by the arrow Y, the state of FIG. 1 results. It is noted here that the state of the defect hole closing material 100 illustrated in FIG. 2A and FIG. 2B is an imaginary state where the coil spring 140 is in an intermediate state between the contracted state and the extended state. Hereinafter, the expression “FIG. 2” alone basically indicates FIG. 2A. In FIG. 2B, dashed lines are imaginary lines schematically representing the outer shape of the defect hole closing material 100 in which the coil spring 140 is in the intermediate state, and a dot-dash line is an imaginary line representing the coil spring 140.

As illustrated in these drawings, an overview of the defect hole closing material 100 is as follows: the defect hole closing material 100 is comprised of a tubular body that has a mesh structure formed of a linear material, the defect hole closing material 100 has a shape in which a substantially middle portion 130 of the tubular body is smaller in tube diameter than other portions of the tubular body, the defect hole closing material 100 has a first tubular portion 110 with a first end 112 and a second tubular portion 120 with an opposite end (second end 122) which are arranged with the substantially middle portion 130 therebetween, the first end 112 and the opposite end being opposite ends of the defect hole closing material 100 in a longitudinal direction of the tubular body. The defect hole closing material 100 is characterized in that the defect hole closing material 100 includes a coil spring 140 (an example of an elastic member) which has opposite ends respectively engaged with a linear material 114 at the first end 112 and a linear material 124 at the second end 122 and which passes through the first tubular portion 110 and the second tubular portion 120 from the first end 112 to the second end 122 via the substantially middle portion 130. The elastic member is not limited to the coil spring 140 and may be a member other than the coil spring 14, provided that the member has elasticity and is capable of achieving effects described later with its elasticity.

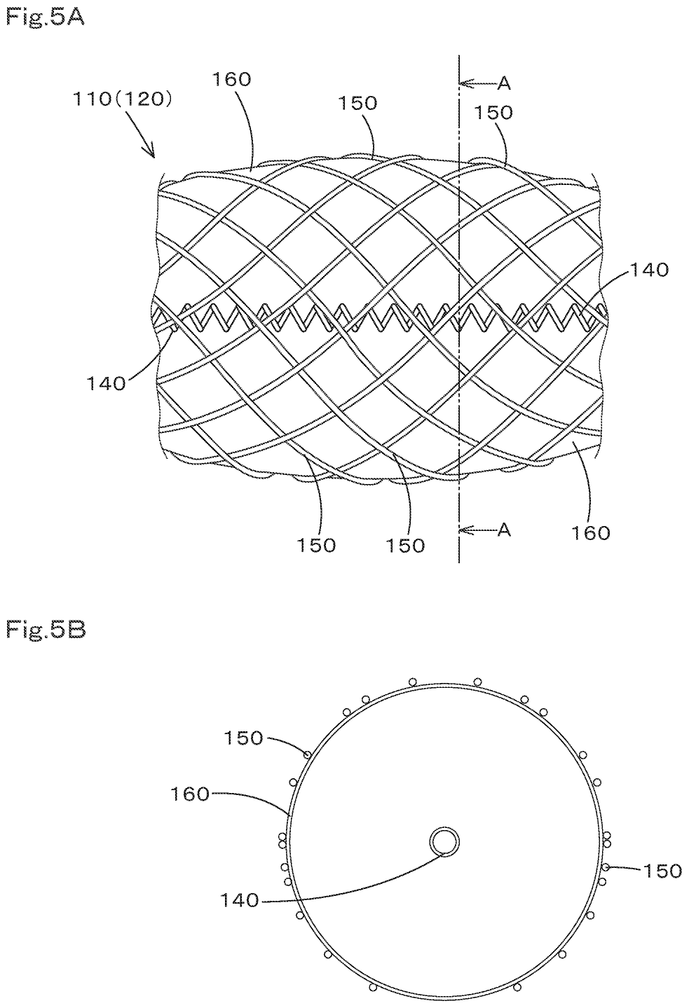

FIG. 5A is a partial side view of the defect hole closing material 100, and FIG. 5B is a cross-sectional view taken along A-A in FIG. 5A. Note that although FIG. 5B is a cross-sectional view of the defect hole closing material 100, FIG. 5B illustrates only cross-sections of the coil spring 140, strands of bioabsorbable fiber 150, and a porous tubular layer 160 and does not illustrate the mesh of the bioabsorbable fiber 150 that is visible from a direction indicated by an arrow A. Furthermore, in FIG. 1 to FIG. 5A and FIG. 5B, the porous tubular layer 160 is illustrated as a transparent material in order to facilitate the understanding of the presence of the coil spring 140 and the mesh of the bioabsorbable fiber 150.

As illustrated in these drawings (particularly FIG. 2), the defect hole closing material 100 is comprised of two tubular bodies (the first tubular portion 110 and the second tubular portion 120) having a mesh structure formed of a bioabsorbable material, and has a shape which is composed of such two tubular bodies and which is called, for example, a sandglass shape, a figure-of-eight shape, a double spindle shape (shape composed of two continuous long rod-like spindle-shaped objects each of which is thick in the middle and thin at both ends), or a peanut shape (outer shape of a peanut shell containing two nuts). The defect hole closing material 100 having such a shape has a shape in which the substantially middle portion 130 is narrowed such that the substantially middle portion 130 is smaller in tube diameter than other portions. That is, the first tubular portion 110 with the first end 112 and the second tubular portion 120 with the second end 122 are arranged with the substantially middle portion 130 therebetween.

In the defect hole closing material 100, the first tubular portion 110 and the second tubular portion 120 are integrally knitted or woven such that the substantially middle portion 130 is smaller in tube diameter than other portions and the defect hole closing material 100 as a whole has a sandglass shape, figure-of-eight shape, double spindle shape, or peanut shape composed of two tubular bodies, although this does not imply limitation. In such a case, the shape of the whole defect hole closing material 100 is formed by, with use of a frame (a three-dimensional paper mold) having such a sandglass shape, figure-of-eight shape, double spindle shape, or peanut shape, knitting or weaving the tubular portions from a strand of the bioabsorbable fiber 150 in conformity with the mold. Further, the defect hole closing material 100 having a sandglass shape, figure-of-eight shape, double spindle shape, or peanut shape composed of two tubular bodies as a whole may be formed in the following manner: the first tubular portion 110 and the second tubular portion 120 are integrally knitted or woven to make a tubular body having a substantially uniform diameter; and then the substantially middle portion 130 is, for example, tied and/or thermally set to obtain a shape in which the substantially middle portion 130 is smaller in tube diameter than other portions; and then the substantially middle portion 130 is untied and/or the thermal setting of the substantially middle portion 130 is discontinued to form the substantially middle portion 130 having a larger tube diameter than the diameter of the coil spring 140, although this does not imply limitation. As will be described in detail later, such a shape makes it possible to achieve the following changes in shape: when the defect hole closing material 100 that is entirely contained in the catheter 300 (in the space defined by the inner wall 310) illustrated in FIG. 3 is pushed from the first tubular portion 110 side in the direction indicated by the arrow Y so that the second tubular portion 120 is pushed out through the opening 320 of the catheter 300, the second tubular portion 120 is released from the space defined by the inner wall 310 of the catheter 300 and the coil spring 140 contracts in the second tubular portion 120, and the state of FIG. 4 results; and when the first tubular portion 110 is further pushed in the direction indicated by the arrow Y, the first tubular portion 110 is released from the space defined by the inner wall 310 of the catheter 300 and the coil spring 140 contracts in the first tubular portion 110, and the state of FIG. 1 results.

Furthermore, the defect hole closing material 100 includes the coil spring 140, which has one end engaged with the first end 112 (for example, held in a loop of the linear material 114 at the first end 112) and the other end engaged with the second end 122 (for example, held in a loop of the linear material 124 at the second end 122) and which is inserted through the first tubular portion 110 and the second tubular portion 120 from the first end 112 to the second end 122 via the substantially middle portion 130. The looped linear material 114 and linear material 124 are formed of the bioabsorbable fiber 150.

As illustrated in FIG. 1, when the coil spring 140 is in the contracted state, the first end 112 and the second end are close to each other with the substantially middle portion 130 therebetween, and the first tubular portion 110 and the second tubular portion 120, as the other portions other than the substantially middle portion 130, increase in tube diameter. It is particularly preferable that, when the coil spring 140 is in the contracted state, the first tubular portion 110 and the second tubular portion 120, as the other portions other than the substantially middle portion 130, increase in tube diameter to a size corresponding to a defect hole to be closed with the defect hole closing material 100.

As illustrated in FIG. 3, when the coil spring 140 is brought into the extended state by, for example, housing the defect hole closing material 100 in the catheter 300, the first end 112 and the second end 122 move away from each other with the substantially middle portion 130 therebetween, and the first tubular portion 110 and the second tubular portion 120, as the other portions, decrease in tube diameter. It is particularly preferable that, when the coil spring 140 is in the extended state, the first tubular portion 110 and the second tubular portion 120 as the other portions decrease in tube diameter to a size corresponding to the catheter 300 in which the defect hole closing material 100 is to be contained.

As described above, by using the coil spring 140 having a diameter smaller than the tube diameter of the substantially middle portion 130, the first end 112 and the second end 122, which are opposite ends of the defect hole closing material 100 in the longitudinal direction of the tubular body, can be brought close to or away from each other. When the coil spring 140 is brought into the contracted state, as illustrated in FIG. 1, the first end 112 and the second end 122 come close to each other and the other portions other than the substantially middle portion 130 (body portion of the first tubular portion 110 and the body portion of the second tubular portion 120) increase in tube diameter. When the coil spring 140 is brought into the extended state, as illustrated in FIG. 3, the first end 112 and the second end 122 move away from each other and the other portions other than the substantially middle portion 130 (body portion of the first tubular portion 110 and the body portion of the second tubular portion 120) decrease in tube diameter. Further, as illustrated in FIG. 4, when the second tubular portion 120 is pushed out of the catheter 300 in the direction indicated by the arrow Y, the second tubular portion 120, which has had its shape restricted by the inner wall 310 of the catheter 300, becomes freely changeable in shape, and only the part of the coil spring 140 that is contained in the second tubular portion 120 contracts and only the body portion of the second tubular portion 120 increases in tube diameter. Furthermore, when the first tubular portion 110 is pushed out of the catheter 300 in the direction indicated by the arrow Y, the first tubular portion 110, which has had its shape restricted by the inner wall 310 of the catheter 300, also becomes freely changeable in shape, and the part of the coil spring 140 that is contained in the first tubular portion 110 also contracts and the body portion of the first tubular portion 110 also increases in tube diameter, as illustrated in FIG. 1.

In the defect hole closing material 100, the first and second tubular portions 110 and 120 and the substantially middle portion 130 are provided with a radiopaque material that is observable in X-ray imaging, as illustrated in FIG. 2B. A method for providing the radiopaque material to the first and second tubular portions 110 and 120 and the substantially middle portion 130 (method for providing the radiopaque material to the bioabsorbable fiber 150) is not particularly limited. Examples thereof include binding a separate member having radiopaque property (a gold tip, a platinum tip, or the like as a metal tip) to the bioabsorbable fiber 150; and applying radiopaque barium sulfate or the like to the bioabsorbable fiber 150.

More specifically, pieces of a radiopaque material 110A are provided substantially at the middle (at or near a part having the maximum diameter) of the first tubular portion 110 in the tubular body longitudinal direction, pieces of a radiopaque material 120A are provided substantially at the middle (at or near a part having the maximum diameter) of the second tubular portion 120 in the tubular body longitudinal direction, and pieces of a radiopaque material 130A are provided at the substantially middle portion 130. Although the number of pieces of the radiopaque material at each position and the positional relations between the pieces of the radiopaque material are not limited, four pieces of the radiopaque material are provided at each position other than the substantially middle portion 130 such that they are spaced apart from each other (with intervals of approximately 90 degrees, for example) in the circumferential direction of the tubular body, and two (which is less than four) pieces of the radiopaque material are provided at the substantially middle portion 130 because the substantially middle portion 130 is short in the tubular body longitudinal direction and has a small diameter. One of the reasons why two or more pieces of a radiopaque material are provided at each position is that an effect which will be described later can be achieved even if one piece of a radiopaque material falls off. One of the reasons why pieces of a radiopaque material are provided such that they are spaced apart from each other in the circumferential direction is to prevent poor visibility that would result from the pieces of the radiopaque material appearing in a gathered manner in X-ray imaging when they are not spaced apart from each other.

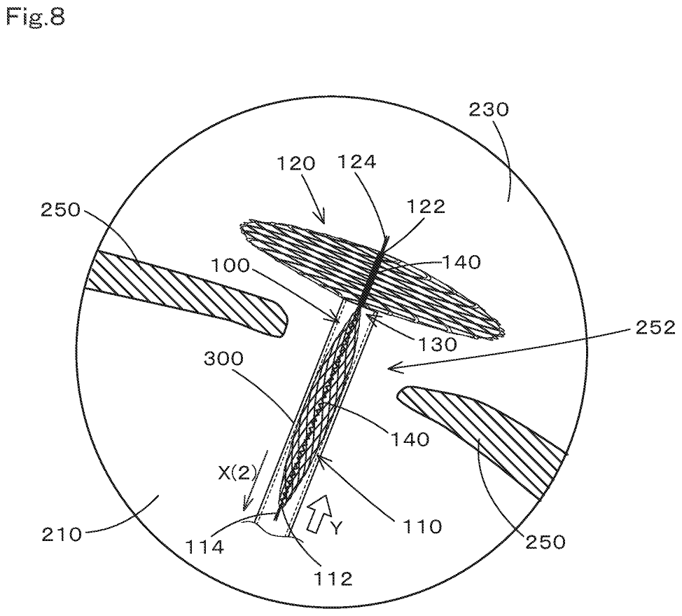

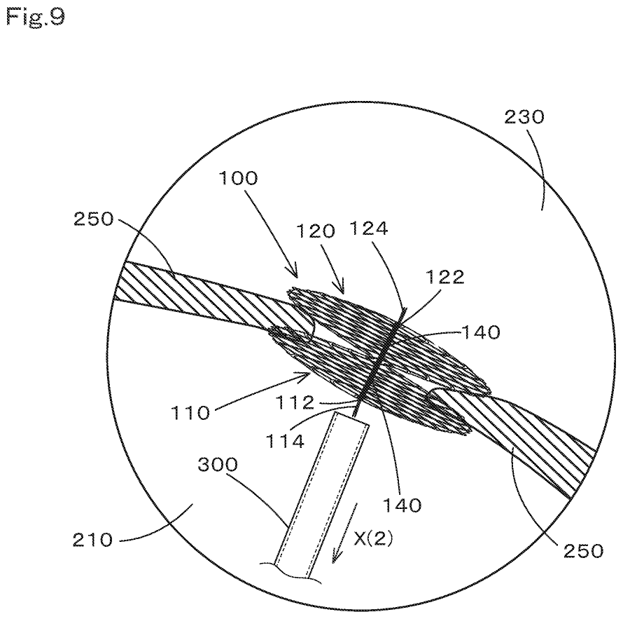

The following section [Usage Embodiments] will discuss a case in which the defect hole closing material 100 is used in catheterization for atrial septal defect. In such a case, it is necessary to ensure that only the body portion of the second tubular portion 120 has increased in tube diameter and the body portion of the first tubular portion 110 has not increased in tube diameter as illustrated in FIG. 4. More specifically, it is necessary to close a defect hole 252 in an atrial septum 250 with the defect hole closing material 100 in the following manner: the defect hole closing material 100 is pushed out of the catheter 300 with an applicator or the like to first expand the second tubular portion 120 located in the left atrium (see FIG. 8) and then expand the first tubular portion 110 located in the right atrium (see FIG. 9), whereby the first tubular portion 110 located in the right atrium and the second tubular portion 120 located in the left atrium come close to each other with the substantially middle portion 130 (defect hole 252) therebetween and the first tubular portion 110 and the second tubular portion 120 increase in tube diameter; and eventually the atrial septum 250 is sandwiched from both sides thereof between the first tubular portion 110 and the second tubular portion 120 to close the defect hole 252. That is, if the second tubular portion 120 and the first tubular portion 110 are both expanded in the left atrium or the right atrium, the defect hole 252 in the atrial septum 250 cannot be closed with the defect hole closing material 100.

In order to avoid such a situation, when the defect hole closing material 100 is used in catheterization for atrial septal defect, a site including the defect hole closing material 100 is X-rayed, and thereby the pieces of the radiopaque material 110A, the pieces of the radiopaque material 120A, and the pieces of the radiopaque material 130A are visually checked in a fluoroscopic X-ray mage. With this, it is possible to visually check (1) the degree to which the first tubular portion 110 has expanded, (2) the degree to which the second tubular portion 120 has expanded, and (3) to what degree the defect hole closing material 100 should be pushed out of the catheter 300 with the applicator or the like in order to expand only the second tubular portion 120 (without expanding the first tubular portion 110). That is, it is possible to visually check to what degree the defect hole closing material 100 should be pushed out of the catheter 300 in order to expand only the second tubular portion 120 (without expanding the first tubular portion 110), because the substantially middle portion 130 is provided with the pieces of the radiopaque material 130A observable in X-ray imaging in addition to the pieces of the radiopaque material 110A of the tubular portion 110 and the pieces of the radiopaque material 120A of the second tubular portion 120.

In this way, it is possible to close the defect hole 252 in the atrial septum 250 with the defect hole closing material 100 by expanding only the second tubular portion 120 in the left atrium and then expanding the first tubular portion 110 in the right atrium while visually checking the degree to which the defect hole closing material 100 has been pushed out of the catheter 300, thereby sandwiching the atrial septum 250 from both sides thereof between the first tubular portion 110 and the second tubular portion 120.

In the defect hole closing material 100, the porous tubular layer 160 formed of nonwoven fabric, a sponge, a film, or a composite thereof, each made of a bioabsorbable material, is disposed on the inner surface of the tubular body. The first tubular portion 110 and the second tubular portion 120 are formed of woven fabric (coarse-woven fabric), knitted fabric, braided fabric, or tubular knitted fabric of the bioabsorbable fiber 150, and are entirely composed of a mesh structure. It should be noted here that the mesh structure is not limited to knitted fabric formed by knitting, but includes a network structure composed of a coarse-woven structure like a window net, as described above. That is, the first tubular portion 110 and the second tubular portion 120 may have a structure called “mesh structure” or a structure called “network structure”. The porous tubular layer 160 is formed of non-woven fabric, a sponge, a film, or a composite thereof, in order to hold a medical agent by application, impregnation, embedding, or the like. Further, the porous tubular layer 160 is not limited to a bioabsorbable material, and may be a non-bioabsorbable material.

As described above, basically the first tubular portion 110, the second tubular portion 120, and the porous tubular layer 160 are all made of a bioabsorbable material except for the coil spring 140, and therefore the entire defect hole closing material 100 except for the coil spring 140 is bioabsorbable. Furthermore, treatment to close a defect hole using the defect hole closing material 100 changing in shape is performed; in this regard, the defect hole closing material 100 employs a material, mesh shape, fiber structure, and fiber cross section that do not damage tissue in a living body even when the shape of the defect hole closing material 100 is thus changed in the living body.

Note that, usually, the coil spring 140 is made of, for example, a nickel-titanium alloy or the like and is not bioabsorbable, but the coil spring 140 may be made of, for example, an alloy based on magnesium (described later) to be bioabsorbable. The use of a bioabsorbable alloy for the coil spring 140 is advantageous in that the coil spring 140 is observable in X-ray imaging, and the use of a non-bioabsorbable alloy is advantageous in that a metallic member does not remain in the body throughout the whole life and therefore an issue of possible problems in the late post-treatment period does not arise. A material that is not observable in X-ray imaging is employed for the coil spring 140 of the defect hole closing material 100.

The bioabsorbable fiber 150 forming the first tubular portion 110 and the second tubular portion 120 is, for example, at least one type selected from polyglycolic acid, polylactides (poly-D-lactide, poly-L-lactide, and poly-DL-lactide), polycaprolactone, glycolic acid-lactide (D-lactide, L-lactide, or DL-lactide) copolymers, glycolic acid-ε-caprolactone copolymers, lactide (D-lactide, L-lactide, or DL-lactide)-ε-caprolactone copolymers, poly(p-dioxanone), glycolic acid-lactide (D-lactide, L-lactide, or DL-lactide)-ε-caprolactone copolymers, and the like. The at least one type of material is used after being processed into any one of the following forms: monofilament yarn, multifilament yarn, twisted yarn, braid, and the like, and is preferably used in the form of a monofilament yarn.

The material for the bioabsorbable fiber 150 may be a biodegradable alloy. Examples of such a biodegradable alloy include alloys based on magnesium as a raw material.

The bioabsorbable fiber 150 has a diameter of about 0.001 mm to 1.5 mm, and fiber diameter and type that are suitable for catheterization in which the defect hole closing material 110 is used are selected. Furthermore, the bioabsorbable fiber 150 may have any of the following cross sections: a circle, an ellipse, and other different shapes (such as a star shape), provided that the in vivo tissue is not damaged. Further, the surface of the bioabsorbable fiber 150 may be treated to have hydrophilicity by plasma discharge, electron beam treatment, corona discharge, ultraviolet irradiation, ozone treatment, or the like.

The first tubular portion 110 and the second tubular portion 120 are formed in the following manner: the bioabsorbable fiber 150 is, for example, braided to form braided fabric using a braiding machine with multiple (for example, 8 or 12) yarn feeders around a silicone rubber tube (not illustrated) having an outer diameter desired as a monofilament yarn or knitted or woven into a tubular mesh structure having a substantially uniform diameter using a circular knitting machine (not illustrated). After the knitting or weaving, as described earlier, the braided fabric or the tubular mesh structure is made narrower in the substantially middle portion 130 with a cord made of the same material as that of the first tubular portion 110 and the second tubular portion 120, and thereby formed into a sandglass shape, figure-of-eight shape, double spindle shape, or peanut shape composed of two tubular bodies. The tube diameters of the first tubular portion 110 and the second tubular portion 120 in a small diameter state are smaller than the inner diameter of the catheter, and the first tubular portion 110 and the second tubular portion 120 in a large diameter state have a size preferable for catheterization for atrial septal defect. For example, the tube diameters of the first tubular portion 110 and the second tubular portion 120 in the large diameter state are about 5 mm to 80 mm, preferably about 15 mm to 25 mm. Furthermore, the lengths of the first tubular portion 110 and the second tubular portion 120 and the density of the mesh structure of the defect hole closing material 100 also have a density preferable for catheterization for atrial septal defect. Note that the first tubular portion 110 and the second tubular portion 120 do not need to have equal tube diameters and do not need to have equal lengths, and the tube diameters and lengths may be changed to suit for catheterization for atrial septal defect.

The bioabsorbable material for the porous tubular layer 160 is not particularly limited, and examples thereof include synthetic absorbable polymers such as polyglycolic acid, polylactides (poly-D-lactide, poly-L-lactide, and poly-DL-lactide), polycaprolactone, glycolic acid-lactide (D-lactide, L-lactide, or DL-lactide) copolymers, glycolic acid-ε-caprolactone copolymers, lactide (D-lactide, L-lactide, or DL-lactide)-ε-caprolactone copolymers, poly(p-dioxanone), and glycolic acid-lactide (D-lactide, L-lactide, or DL-lactide)-ε-caprolactone copolymers. Such materials may be used alone or in combination of two or more. Among those listed above, at least one type selected from the group consisting of polyglycolic acid, lactide (D-lactide, L-lactide, or DL-lactide)-ε-caprolactone copolymers, glycolic acid-ε-caprolactone copolymers, and glycolic acid-lactide (D-lactide, L-lactide, or DL-lactide)-ε-caprolactone copolymers is preferable because of appropriate degradation behavior, and the porous tubular layer 160 is formed of non-woven fabric, a sponge, a film, or a composite thereof. In particular, an example of a preferred embodiment is non-woven fabric.

The material for the porous tubular layer 160 may be a biodegradable alloy. Examples of such a biodegradable alloy include alloys based on magnesium as a raw material.

When the porous tubular layer 160 is formed of non-woven fabric, the porous tubular layer 160 may be treated to have hydrophilicity. The treatment to impart hydrophilicity is not particularly limited, and is, for example, plasma treatment, glow discharge treatment, corona discharge treatment, ozone treatment, surface grafting treatment, ultraviolet irradiation treatment, or the like. Among those listed above, plasma treatment is preferable because the plasma treatment can dramatically improve water absorption rate without changing the appearance of the non-woven fabric layer. Note that the porous tubular layer 160 may be a sponge layer or a film layer or may be a composite layer composed of non-woven fabric and a sponge layer, a composite layer composed of non-woven fabric and a film layer, a composite layer composed of a sponge layer and a film layer, or a composite layer composed of non-woven fabric, a sponge layer, and a film layer.

The porous tubular layer 160 is configured to have, held thereon, a medical agent suitable for catheterization for atrial septal defect.

As has been described, the defect hole closing material 100 according to the present embodiment includes the following features.

(First feature) The defect hole closing material 100 has a sandglass shape, figure-of-eight shape, double spindle shape, or peanut shape that is thin in the substantially middle portion 130 and that is comprised of the first tubular portion 110 and the second tubular portion 120.
(Second feature) The defect hole closing material 100 includes the coil spring 140 which has one end engaged with the first end 112 (held in the looped linear material 114 at the first end 112), which has the other end engaged with the second end 122 (held in the looped linear material 124 at the second end 122), and which passes through the first tubular portion 110 and the second tubular portion 120 from the first end 112 to the second end 122 via the substantially middle portion 130.
(Third feature) The defect hole closing material 100 is comprised of the first tubular portion 110, the second tubular portion 120, the coil spring 140 (in cases where the coil spring 140 is made of a magnesium-based alloy), and the porous tubular layer 160, and these components are all made of a bioabsorbable material (the coil spring 140 does not need to be bioabsorbable).

With the first feature and the second feature, with regard to the defect hole closing material 100 contained in the catheter 300, when the second tubular portion 120 is pushed out of the catheter 300, the second tubular portion 120, which has had its shape restricted by the inner wall 310 of the catheter 300, becomes freely changeable in shape, and only the part of the entire coil spring 140 that is contained in the second tubular portion 120 contracts and only the body portion of the second tubular portion 120 increases in tube diameter, and, furthermore, when the first tubular portion 110 is pushed out of the catheter 300, the first tubular portion 110, which has had its shape restricted by the inner wall 310 of the catheter 300, also becomes freely changeable in shape, and the part of the entire coil spring 140 that is contained in the first tubular portion 110 also contracts and the body portion of the first tubular portion 110 also increases in tube diameter.

In particular, the defect hole closing material 100 is suitable for catheterization for atrial septal defect because it provides the following effects.

(First effect) The defect hole closing material 100 can be set in the catheter 300 by extending the entire coil spring 140 to cause the defect hole closing material 100 to have a smaller tube diameter than the inner diameter of the catheter 300.

(Second effect) The defect hole closing material 100 is set in the catheter 300 and sent to the position of a hole in the atrial septum. When the first end 112 is pushed with an applicator or the like in a living body and thereby the second tubular portion 120 is pushed out of the catheter 300 into the living body, the part of the coil spring 140 in the second tubular portion 120 contracts and the body portion of the second tubular portion 120 increases in tube diameter, and, when the first end 112 is further pushed with the applicator or the like and thereby the first tubular portion 110 is pushed out of the catheter 300 into the living body, the part of the coil spring 140 in the first tubular portion 110 also contracts and the body portion of the first tubular portion 110 also increases in tube diameter. With this, the first tubular portion 110 located in the right atrium and the second tubular portion 120 located in the left atrium come close to each other with the substantially middle portion 130 therebetween, thereby making it possible to close the hole in the atrial septum.

(Third effect) The materials (excluding the coil spring 140 in some cases) for the defect hole closing material 100 are all bioabsorbable, and therefore are eventually absorbed by the living body. This substantially eliminates the likelihood that problems will occur in the late post-treatment period.

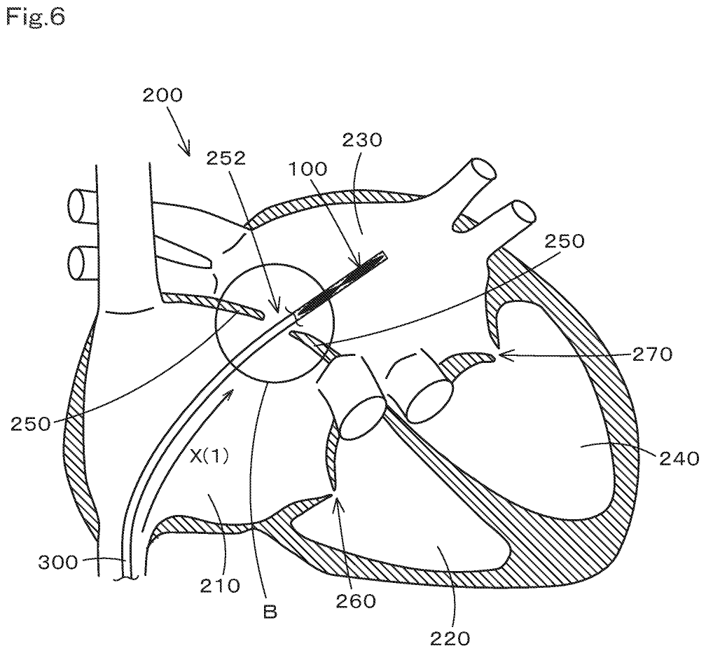

For easy understanding of such effects, the following description discusses a case in which the defect hole closing material 100 is used in catheterization for atrial septal defect, with reference to FIG. 6 to FIG. 9.

### Usage Embodiments

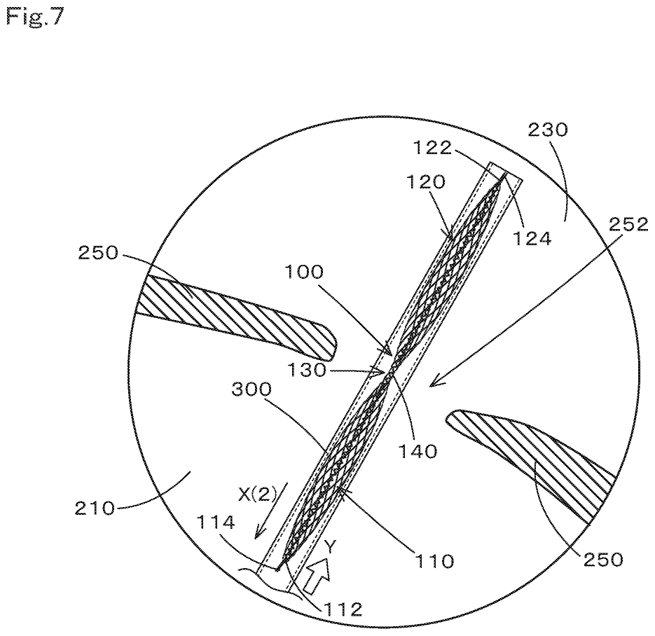

FIG. 6 is a conceptual view in which the defect hole closing material 100 is used in catheterization for atrial septal defect, and FIG. 7 to FIG. 9 are enlarged views of a part B in FIG. 6 and illustrate the procedure of the catheterization. Note that the following description only discusses matters specific to the usage embodiments of the defect hole closing material 100 according to the present embodiment, and does not specifically discuss general matters because these are the same as those of known catheterization for atrial septal defect.

As illustrated in FIG. 6, a heart 200 of a human has two atria and two ventricles: a right atrium 210 connected to the superior vena cava and the inferior vena cava to receive venous blood from the whole body; a right ventricle 220 connected to the right atrium 210 via a pulmonary artery and a tricuspid valve 260 to send venous blood to the lungs; a left atrium 230 connected to a pulmonary vein to receive arterial blood from the lungs; and a left ventricle 240 connected to the left atrium 230 via the aorta and a mitral valve 270 to send arterial blood to the whole body. Atrial septal defect is a defect in which there is a defect hole 252 in an atrial septum 250 separating the right atrium 210 and the left atrium 230. Note that, in FIG. 6, an end portion of the catheter 300 is represented by an imaginary line and the defect hole closing material 100 contained in the catheter 300 is represented by a solid line for easy understanding.

First, outside the living body, the defect hole closing material 100, which expands to a size appropriate for the defect hole 252, is pulled such that the first end 112 and the second end 122 are directed away from each other, thereby extending the entire coil spring 140 and causing the defect hole closing material 100 to have a smaller tube diameter than the inner diameter of the catheter 300, and the defect hole closing material 100 is set in the catheter 300. The catheter 300 containing the defect hole closing material 100 is inserted through a femoral vein (see FIG. 3) and is moved in the direction indicated by an arrow X(1) to pass through the defect hole 252 from the right atrium 210, and the catheter 300 containing the defect hole closing material 100 is brought close to the left atrium 230 side.

As illustrated in FIG. 6 and FIG. 7, the catheter 300 containing the defect hole closing material 100 is stopped at a position where the substantially middle portion 130 of the defect hole closing material 100 substantially corresponds to the defect hole 252. In the living body, when the second tubular portion 120 is pushed out of the catheter 300 with an applicator or the like in the direction indicated by the arrow Y, the second tubular portion 120, which has had its shape restricted by the inner wall 310 of the catheter 300, becomes freely changeable in shape, and only the part of the coil spring 140 that is contained in the second tubular portion 120 contracts and only the body portion of the second tubular portion 120 increases in tube diameter as illustrated in FIG. 8.

In so doing, by visually checking the position of the radiopaque material 130A in a fluoroscopic X-ray image obtained by X-ray imaging of the site that includes the defect hole closing material 100, it is possible to know the degree to which the defect hole closing material 100 should be pushed out of the catheter 300 with the applicator or the like to achieve a state in which only the second tubular portion 120 is expanded and the first tubular portion 110 is not expanded. More specifically, since the catheter 300 itself appears in the fluoroscopic X-ray image obtained by X-ray imaging, it is only necessary that the second tubular portion 120 be pushed out of the catheter 300 in the direction indicated by the arrow Y with the applicator or the like until the radiopaque material 130A reaches the vicinity of the exit of the catheter 300.

Furthermore, when the first tubular portion 110 is pushed out of the catheter 300 with the applicator or the like in the direction indicated by the arrow Y, the first tubular portion 110, which has had its shape restricted by the inner wall 310 of the catheter 300, also becomes freely changeable in shape, and the part of the coil spring 140 that is contained in the first tubular portion 110 also contracts and the body portion of the first tubular portion 110 also increases in tube diameter, as illustrated in FIG. 9.

That is, when the defect hole closing material 100 is pushed out of the catheter 300 with an applicator or the like, the second tubular portion 120 located in the left atrium expands first, and then the first tubular portion 110 located in the right atrium expands. It follows that the first tubular portion 110 located in the right atrium and the second tubular portion 120 located in the left atrium come close to each other with the substantially middle portion 130 (defect hole 252) therebetween, and that the first tubular portion 110 and the second tubular portion 120 increase in tube diameter. Eventually, as illustrated in FIG. 9, the first tubular portion 110 and the second tubular portion 120 sandwich the atrial septum 250 from both sides, thereby making it possible to close the defect hole 252 in the atrial septum 250 with the defect hole closing material 100.

After that, the catheter 300 is moved in the direction indicated by an arrow X(2) to take the catheter 300 out of the living body, thereby completing the treatment. With this, in the living body (technically, in the vicinity of the defect hole 252), the defect hole closing material 100 entirely made of a bioabsorbable material (the coil spring 140 is excluded in some cases) is placed. As such, since all the materials for the defect hole closing material 100 placed in the living body are bioabsorbable (the coil spring 140 is excluded in some cases), the defect hole closing material 100 is eventually absorbed by the living body. This substantially eliminates the likelihood that problems will occur in the late post-treatment period.

Note that, when the defect hole closing material 100 does not include the coil spring 140, it is necessary to fix the defect hole closing material 100 in the form illustrated in FIG. 9 before placing the defect hole closing material 100 in the living body. One way of achieving this has been to, for example, employ thermally fusible bioabsorbable fiber 150 and thermally set the bioabsorbable fiber 150 within the living body. In contrast, with regard to the defect hole closing material 100, the defect hole closing material 100 can be fixed in the form illustrated in FIG. 9 with use of the coil spring 140, and thus is advantageous.

As has been described, since the defect hole closing material 100 according to the present embodiment is entirely made of a bioabsorbable material (the coil spring 140 is excluded in some cases) and is eventually absorbed by the living body, there is no or little likelihood that problems will occur in the late posts-treatment period. Furthermore, the presence of the coil spring 140 allows the defect hole closing material 100 to easily change in tube diameter, and therefore the defect hole closing material 100 can be easily set in the catheter by reducing the tube diameter of the defect hole closing material 100. Furthermore, since the defect hole closing material 100 includes the coil spring 140, only by pushing the defect hole closing material 100 out of the catheter 300 at the position of the defect hole, it is possible to easily change the defect hole closing material 100 such that the defect hole closing material 100 increases in tube diameter and the two tubular bodies come close to each other and possible to fix the form of the defect hole closing material 100, thereby closing the defect hole in the atrial septum.

Note that the embodiments disclosed herein should be considered as examples in all aspects and should not be construed as limitations. The scope of the present invention is defined not by the foregoing description but by the claim(s), and is intended to include all modifications within the scope of the claim(s) and their equivalents.

### INDUSTRIAL APPLICABILITY

The present invention is suitable for use as a medical material which is set in a catheter to treat a defect hole in a biological tissue, and is particularly preferable in that the medical material is capable of being released and placed at a treatment site, enables less invasive treatment, and is unlikely to cause a problem in the late post-treatment period even when the medical material remains in the body.

### DESCRIPTION OF THE REFERENCE NUMERAL

100 Medical Material (Occluder)
110 First tubular portion
112 First end
120 Second tubular portion
122 Second end
130 Substantially middle portion
110a, 120a, 130a Radiopaque material
140 Coil spring
150 Bioabsorbable fiber
160 Porous tubular layer
200 Heart
250 Atrial septum
252 Defect hole
300 Catheter

1. A medical material comprised of a tubular body that has a mesh structure formed of a linear material, wherein:
the medical material has a shape in which a substantially middle portion of the tubular body is smaller in tube diameter than other portions of the tubular body;
the medical material has a first tubular portion with a first end and a second tubular portion with an opposite end which are arranged with the substantially middle portion therebetween, the first end and the opposite end being opposite ends of the medical material in a longitudinal direction of the tubular body; and
the first and second tubular portions and the substantially middle portion are provided with a radiopaque material.
